(19) United States
(12) Patent Application Publication
(10) Pub. No.: US 2007/0073141 A1
(43) Pub. Date: Mar. 29, 2007

(54) BREATH HOLDING MR IMAGING METHOD, MRI APPARATUS, AND TOMOGRAPHIC IMAGING APPARATUS

(76) Inventors: Yuji Iwadate, Tokyo (JP); Atsushi Nozaki, Tokyo (JP); Tetsuji Tsukamoto, Tokyo (JP); Hiroyuki Kabasawa, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 11/531,973

(22) Filed: Sep. 14, 2006

(30) Foreign Application Priority Data
Sep. 15, 2005 (JP) 2005-267763

(51) Int. Cl. A61B 5/05

(52) U.S. Cl. 600/410

(57) ABSTRACT

When two or more run of breath holding imaging with breathing interval therebetween is conducted, the present invention allows the slice position to follow the positional displacement of organ in each run. The present invention comprises an image capturing step for the navigator for imaging an MR image having the imaging area including the diaphragm during breath holding, a diaphragm position obtaining step for obtaining the diaphragm position by analyzing the navigator image, an image capturing step for the actual imaging for capturing an MR image of a desired slice during breath holding, and a breathing interval step for releasing respiration, in which these steps are iteratively repeated for twice or more. The slice position of second session or later is made to be a position which is set such that the slice position of first run is compensated for by the difference between the diaphragm position of the first run and the diaphragm position of the second run or later.

### CROSS REFERENCE TO RELATED APPLICATIONS

This application claims the benefit of Japanese Application No. 2005-267763 filed Sep. 15, 2005.

### BACKGROUND OF THE INVENTION

The present invention is related to a breath holding MR (magnetic resonance) imaging method, MRI (magnetic resonance imaging) apparatus, breath holding tomographic imaging method, and tomographic imaging apparatus, and more specifically to a breath holding MR (magnetic resonance) imaging method, MRI (magnetic resonance imaging) apparatus, breath holding tomographic imaging method, and tomographic imaging apparatus, allowing the slice position to follow the displacement of organs between two or more repetitive imaging sessions of breath holding imaging with breath releasing time therebetween.

So far, when two or more breath holding imaging sessions with breath releasing interval therebetween, there has been proposed a method by observing the position of diaphragm for directing a breath holding at the moment where the diaphragm is at the same position (for example, patent document 1 cited below).

On the other hand, when a non breath holding imaging (an imaging session in which the breath is not held) is conducted, there has been proposed a method in which, by detecting the displacement of a subject caused by the respiration, the slice position is moved so as to follow the amount of displacement (for example, patent document 2 cited below).

Patent document 1: JP-A-2004-508859 (claim 8, [0008])

Patent document 2: JP-A-2004-305454 (claim 6, [0048]-[0050])

In the patent document 1 cited above, a technique for directing a breath holding at the moment when the diaphragm is at the same position is not specifically disclosed. If the subject is signaled to hold the breath at the moment when the diaphragm is at the same position, the fluctuation of breath holding timing persists as long as the breath is voluntarily held by the will of the subject, so that the position of diaphragm will be displaced at the actual timing of breath holding. This poses a problem that the position of organs with respect to the slice position vary every time.

On the other hand, in the patent document 2 cited above, the slice position adjustment is mainly applied to the non breath holding imaging session, and not to the breath holding imaging session. However, when there are two or more repetitive breath holding imaging sessions with breathing interval therebetween, the fluctuation of breath holding timing in each session may persist, and finally the position of diaphragm will vary at the moment when the breath is actually held. This also poses a problem even with the patent document 2 that the position of organs with respect to the slice position may vary each time of the breath holding imaging sessions.

### SUMMARY OF THE INVENTION

Therefore, an object of the present invention is to provide a breath holding MR imaging method, MRI apparatus, breath holding tomographic imaging method, and tomographic imaging apparatus, which allow the slice position to follow the positional displacement of organs each time when two or more breath holding imaging sessions is conducted with the breath releasing interval therebetween.

In a first aspect of the present invention, the present invention provides a breath holding MR imaging method comprising: an image capturing step for navigator for imaging an MR image having the imaging area including the diaphragm while holding breath; a diaphragm position obtaining step for obtaining the position of diaphragm by analyzing the navigator image; an image capturing step for the actual imaging for imaging an MR image of a desired slice while holding breath; and a breath releasing interval step for releasing respiration, wherein the steps are iteratively repeated two or more times in this order, and wherein the slice position of second session or later is made to be a position which is set such that the slice position of first run is compensated for by the difference between the diaphragm position of the first run and the diaphragm position of the second run or later.

The breath holding MR imaging method in accordance with the first aspect as have been described above, the position of diaphragm in breath holding is detected to compensate for the slice position in correspondence with the position of diaphragm. In this manner when two or more breath holding imaging session are conducted with breath releasing interval therebetween, the slice position may follow the positional displacement of organs caused by the fluctuation of breath holding timing in each session.

In a second aspect of the present invention, the present invention provides a breath holding MR imaging method in accordance with the first aspect, in which the imaging area of the image capturing step for the navigator is in the form of a line extending along with the body axis.

The displacement of diaphragm due to the respiration is three-dimensional, but the primary direction of displacement lies in the direction of body axis. It is therefore sufficiently effective to obtain the displacement in the direction of body axis.

For this reason, the breath holding MR imaging method in accordance with the second aspect of the present invention, the imaging area in the image capturing step for the navigator is in the form of a line in the direction of body axis. This needs only the minimal data acquisition and computation.

In a third aspect of the present invention, the present invention provides a breath holding MR imaging method, in which prior to the image capturing step for the actual imaging in the breath holding MR imaging method in accordance with either the first or second aspect of the present invention, a recovery step is provided for waiting for one second or more for the residual lateral magnetized component to be recovered to vertical magnetization.

The breath holding MR imaging method in accordance with the third aspect above of the present invention, the affection of the image capturing step for the navigator to the image capturing step for the actual imaging is prevented.

In a fourth aspect of the present invention, the present invention provides a breath holding MR imaging method, in which prior to the image capturing step for the actual imaging in the breath holding MR imaging method in accordance with any one of the first to third aspect of the present invention, a fast recovery step is provided for forcibly recovering the residual lateral magnetized component to vertical magnetization.

In the breath holding MR imaging method in accordance with the fourth aspect above of the present invention, the affection of the image capturing step for the navigator to the image capturing step for the actual imaging is prevented.

In a fifth aspect of the present invention, the present invention provides a breath holding MR imaging method in accordance with any one of the first to fourth aspect in which the image capturing step for the navigator and the image capturing step for the actual imaging use the reverse centric view ordering.

In the breath holding MR imaging method in accordance with the fifth aspect of the present invention, the affection of the image capturing step for the navigator to the image capturing step for the actual imaging is prevented.

In a sixth aspect of the present invention, the present invention provides a breath holding MR imaging method in accordance with any one of the first to fifth aspect of the present invention, in which a contrast MR angiography of abdomen imaging is conducted in the image capturing step for the actual imaging.

In the breath holding MR imaging method in accordance with the sixth aspect above of the present invention, the imaging position with respect to the organs is the same for every time, a correct time intensity curve can be obtained. In addition a correct subtraction image can be obtained.

In a seventh aspect of the present invention, the present invention provides a breath holding MR imaging method in accordance with any one of the first to fifth aspect of the present invention, in which a multi-phase imaging of liver (arterial phase, portal vein phase, equilibrium phase) is conducted in the image capturing step for the actual imaging.

In the breath holding MR imaging method in accordance with the seventh aspect of the present invention, the imaging position with respect to the liver is the same for every time, so that the comparison between phases can be correctly performed.

In a eighth aspect of the present invention, the present invention provides a breath holding MR imaging method in accordance with any one of the first to fifth aspect of the present invention, in which a sensitivity compensation imaging is conducted using a body coil in a first run of the image capturing step for the actual imaging, and image capturing for the actual image is conducted in second run or later of the image capturing step for the actual imaging.

In the breath holding MR imaging method in accordance with the eighth aspect of the present invention, the slice position at the time of sensitivity compensation image capturing may be identical to the slice position at the time of image capturing for the actual image, with respect to the organ.

In a ninth aspect of the present invention, the present invention provides a breath holding MR imaging method in accordance with any one of the first to fifth aspect of the present invention, in which an image is captured for a reference image in a first run of the image capturing step for the actual imaging, and an image is captured for the parallel imaging in a second or later run of the image capturing step for the actual imaging.

In the breath holding MR imaging method in accordance with the ninth aspect of the present invention, the slice position of the reference image and the slice position at the time of parallel imaging may be the same with respect to the organ.

In a tenth aspect of the present invention, the present invention provides a breath holding MR imaging method in accordance with any one of the first to fifth aspect of the present invention, in which an image is captured for image fusion by varying the imaging condition for each time in the image capturing step for the actual imaging.

In the breath holding MR imaging method in accordance with the tenth aspect of the present invention, the misregistration can be eliminated.

In an eleventh aspect of the present invention, the present invention provides a breath holding MR imaging method in accordance with any one of the first to fifth aspect of the present invention, in which an image capturing for a dispersion image is conducted in the image capturing step for the actual image.

In the breath holding MR imaging method in accordance with the eleventh aspect of the present invention, any distortion may be omitted when NEX is increased.

In a twelfth aspect of the present invention, the present invention provides an MRI apparatus, comprising: an image capturing means for a navigator for capturing MR images having an image area including a diaphragm while holding the breath; a diaphragm position obtaining means for obtaining the diaphragm position by analyzing the navigator image; an image capturing means for the actual imaging for capturing MR images of a desired slice while holding the breath; a controller means for repetitively operating the means for two or more times with a breathing interval therebetween; and a compensator means for making the position, which is set such that the slice position of a first run is compensated for by the difference between the diaphragm position of the first run and the diaphragm position of the second run or later, to be the slice position of a second run or later.

In the MRI apparatus in accordance with the twelfth aspect above of the present invention, the breath holding MR imaging method in accordance with the first aspect of the present invention can be suitably embodied.

In a thirteenth aspect of the present invention, the present invention provides an MRI apparatus in accordance with the MRI apparatus of the twelfth aspect of the present invention, in which the imaging area of the image capturing step for the navigator is in the form of a line extending along with the body axis.

In the MRI apparatus of the above thirteenth aspect of the present invention, the breath holding MR imaging method in accordance with the second aspect of the present invention can be suitably embodied.

In a fourteenth aspect of the present invention, the present invention provides an MRI apparatus in accordance with the MRI apparatus of the twelfth or the thirteenth aspect, in which a recovery time for waiting for one second or more for the residual lateral magnetized component to be recovered to vertical magnetization prior to operating the image capturing means for the actual imaging.

In the MRI apparatus in accordance with the fourteenth aspect above of the present invention, the breath holding MRI imaging method in accordance with the third aspect of the present invention can be suitably embodied.

In a fifteenth aspect of the present invention, the present invention provides an MRI apparatus in accordance with the MRI apparatus of any one of the twelfth to the fourteenth aspect, in which a fast recovery means for forcibly recovering the residual lateral magnetized component to vertical magnetization is operated prior to operating the image capturing means for the actual imaging.

In the MRI apparatus in accordance with the fifteenth aspect above of the present invention, the breath holding MRI imaging method in accordance with the fourth aspect of the present invention can be suitably embodied.

In a sixteenth aspect of the present invention, the present invention provides an MRI apparatus in accordance with the MRI apparatus of any one of the twelfth to the fifteenth aspect of the present invention, in which the image capturing step for the navigator and the image capturing step for the actual imaging use the reverse centric view ordering.

In the MRI apparatus in accordance with the sixteenth aspect above of the present invention, the breath holding MR imaging method in accordance with the fifth aspect of the present invention can be suitably embodied.

In a seventeenth aspect of the present invention, the present invention provides a breath holding MR imaging method, using the MRI apparatus in accordance with the twelfth to the sixteenth aspect above of the present invention, for conducting a contrast MR angiography of abdomen imaging.

In the breath holding MR imaging method in accordance with the seventeenth aspect of the present invention, the breath holding MR imaging method in accordance with the sixth aspect of the present invention can be suitably embodied.

In a eighteenth aspect of the present invention, the present invention provides a breath holding MR imaging method by means of the MRI apparatus in accordance with any one of the twelfth to the sixteenth aspect of the present invention, in which the multi-phase image capturing of the liver is conducted.

In the breath holding MR imaging method in accordance with the eighteenth aspect above of the present invention, the breath holding MR imaging method in accordance with the seventh aspect of the present invention can be suitably embodied.

In a nineteenth aspect of the present invention, the present invention provides a breath holding MR imaging method, using the MRI apparatus in accordance with any one of the twelfth to the sixteenth aspect of the present invention, for conducting a sensitivity compensation imaging using a body coil in a first run of the image capturing step for the actual imaging, and conducting image capturing for the actual image in second run or later of the image capturing step for the actual imaging.

In the breath holding MR imaging method in accordance with the nineteenth aspect above of the present invention, the breath holding MR imaging method in accordance with the eighth aspect of the present invention can be suitably embodied.

In a twentieth aspect of the present invention, the present invention provides a breath holding MR imaging method, using the MRI apparatus in accordance with any one of the twelfth to the sixteenth aspect of the present invention, for conducting reference image capturing in a first run of the image capturing step for the actual imaging, and conducting parallel image capturing in a second run or later of the image capturing step for the actual imaging.

In the breath holding MR imaging method in accordance with the twentieth aspect of the present invention, the breath holding MR imaging method in accordance with the ninth aspect of the present invention can be suitably embodied.

In a twenty-first aspect of the present invention, the present invention provides a breath holding MR imaging method, using the MRI apparatus in accordance with any one of the twelfth to the sixteenth aspect of the present invention, for conducting an image capturing for image fusion by varying the imaging condition for each time.

In the breath holding MR imaging method in accordance with the twenty-first aspect above of the present invention, the breath holding MR imaging method in accordance with the tenth aspect of the present invention can be suitably embodied.

In a twenty-second aspect of the present invention, the present invention provides a breath holding MR imaging method, using the MRI apparatus in accordance with any one of the twelfth to the sixteenth aspect of the present invention, for conducting an image capturing for a dispersion image.

In the breath holding MR imaging method in accordance with the twenty-second aspect above of the present invention, the breath holding MR imaging method in accordance with the eleventh aspect of the present invention can be suitably embodied.

In a twenty-third aspect of the present invention, the present invention provides a breath holding tomographic imaging method comprising: an image capturing step for navigator for capturing a tomographic image having an imaging area including the diaphragm while holding the breath; a diaphragm position obtaining step for obtaining the diaphragm position by analyzing the navigator image; an image capturing step for the actual imaging for capturing a tomographic image of a desired slice while holding the breath; and a breathing step for releasing the breath, wherein the steps are iteratively repeated for twice or more in this order, and wherein the slice position of second session or later is made to be a position which is set such that the slice position of first run is compensated for by the difference between the diaphragm position of the first run and the diaphragm position of the second run or later.

In the breath holding tomographic imaging method in accordance with the twenty-third aspect above of the present invention, the diaphragm position is detected while holding the breath, and the slice position is compensated for accordingly in correspondence with the detected diaphragm position. When two or more successive breath holding imaging with a breathing interval therebetween is conducted, the slice position can follow the displacement of the organ in each run.

In a twenty-fourth aspect of the present invention, the present invention provides a tomographic imaging apparatus, comprising: an imaging means for a navigator which captures a tomographic image having an imaging area including the diaphragm in the direction of body axis while holding the breath; a diaphragm position obtaining means for obtaining the diaphragm position by analyzing the navigator image; an imaging means for the actual imaging for capturing a tomographic image of a desired slice while holding the breath; a controller means for repetitively operating the means for twice or more with a breathing interval therebetween; and a compensator means for making the position, which is set such that the slice position of a first run is compensated for by the difference between the diaphragm position of the first run and the diaphragm position of the second run or later, to be the slice position of a second run or later.

In the tomographic imaging apparatus in accordance with the twenty-fourth aspect above of the present invention, the breath holding tomographic imaging method in accordance with the twenty-third aspect of the present invention can be suitably embodied. As can be seen from the foregoing the tomographic imaging apparatus may include an X-ray CT apparatus, in addition to an MRI apparatus.

In accordance with the breath holding MR imaging method, MRI apparatus, breath holding tomographic imaging method, and tomographic imaging apparatus of the present invention, the positional displacement of an organ can be followed by the slice position when the breath holding imaging for twice or more with the breathing interval therebetween.

The breath holding MR imaging method, MRI apparatus, breath holding tomographic imaging method, and tomographic imaging apparatus in accordance with the present invention can be used for obtaining tomographic images of a subject by iteratively repeating twice or more the breath holding image capturing session with a breathing interval therebetween.

Further objects and advantages of the present invention will be apparent from the following description of the preferred embodiments of the invention as illustrated in the accompanying drawings.

### BRIEF DESCRIPTION OF THE DRAWINGS

FIG. 1 is a schematic block diagram illustrating the overview of an MRI apparatus in accordance with first preferred embodiment of the present invention;

FIG. 2 is a flow diagram illustrating the scanning process iteratively repeating the breath holding in accordance with first preferred embodiment of the present invention;

FIG. 3 is a timing chart illustrating the breath holding imaging in accordance with first preferred embodiment of the present invention; and

FIGS. 4a, 4b, 4c, and 4d are schematic diagrams illustrating the displacement of organ due to the timing difference of breath holding.

### DETAILED DESCRIPTION OF THE INVENTION

The present invention will be described in greater details with reference to the preferred embodiments depicted in the accompanying drawings. It should be noted here that the preferred embodiments shown are not to be considered to limit the present invention.

### First Embodiment

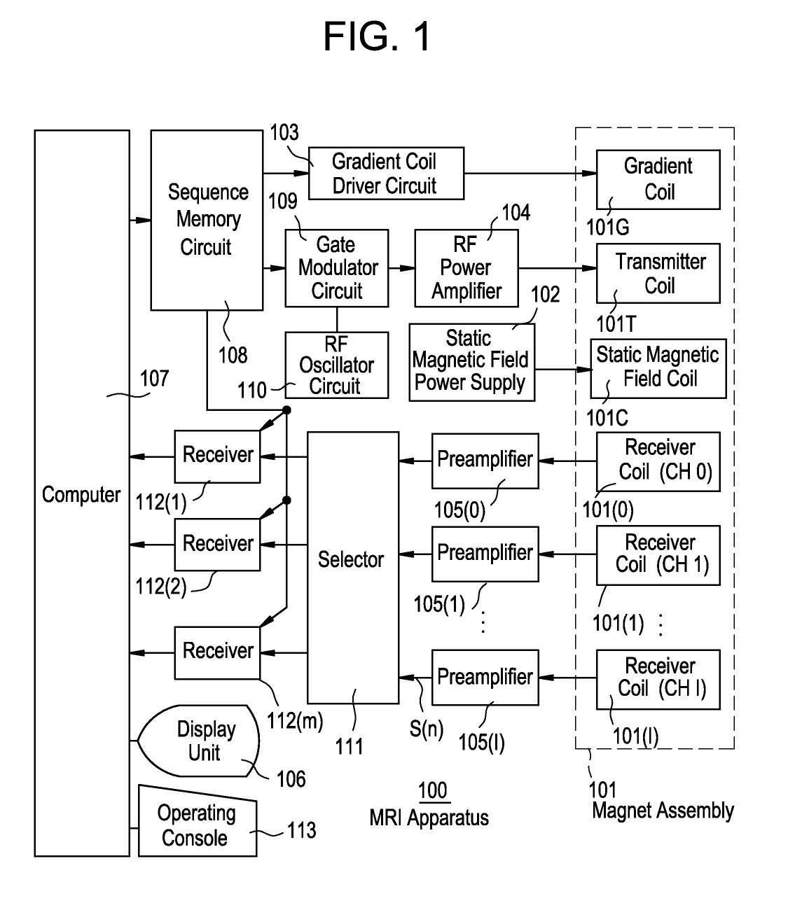

Now referring to FIG. 1, there is shown a schematic block diagram indicating an MRI apparatus 100 in accordance with a first preferred embodiment of the present invention.

In this MRI apparatus 100, a magnet assembly 101 includes a central void (bore) for carrying therein a subject. As surrounding the bore, a static magnetic field coil 101C for applying a constant static field to the subject, a gradient coil 101G for generating a gradient field in the direction of X—, Y—, and Z-axes, a transmitter coil 101T for transmitting RF pulses for exciting the spins of atomic nuclei within the subject, a plurality of receiver coils 101(0) . . . 101(T) for receiving NMR signals from the subject, are arranged.

The transmitter coil 101T and the receiver coil 101(0) are body coils, while the receiver coils 101(1) . . . 101(T) are surface coils.

The static magnetic field coil 101C, the gradient coil 101G, the transmitter coil 101T are each connected to a static magnetic field power supply 102, a gradient coil driver circuit 103, and an RF power amplifier 104, respectively. The receiver coils 101(0) . . . 101(T) are each connected to preamplifiers 105(0) . . . 105(I).

A permanent magnet can be used instead of the static magnetic field coil 101C.

A sequence memory circuit 108, under the control of a computer 107, operates the gradient coil driver circuit 103 based on a pulse sequence stored therein to generate a gradient magnetic field from the gradient coil 101G, and operates a gate modulator circuit 109 to modulate the carrier output signals of an RF oscillator circuit 110 to a pulsive signals having a predefined timing, a predetermined envelope shape, and a predetermined phase to apply to the RF power amplifier 104 as RF pulses, and the RF power amplifier 104 amplifies the power output and applies thus amplified power to the transmitter coil 101T.

A selector 111 transmits, to m receivers 112(1) . . . 112(m), the NMR signals received by the receiver coils 101 (0) . . . 101 (I) and then amplified by the preamplifiers 105(0) . . . 105(I). This solution is for changing arbitrarily the combination of the receiver coils 101(0) . . . 101(T) with the receivers 112(1) . . . 112(m).

The receivers 112(1) . . . 112(m) converts the NMR signals into digital ones to input into the computer 107.

The computer 107 reads the digital signals from the receivers 112 to process on it to generate an MR image. The computer 107 also performs the overall machine management such as receiving information input from a console 113.

A display unit 106 displays images and messages thereon.

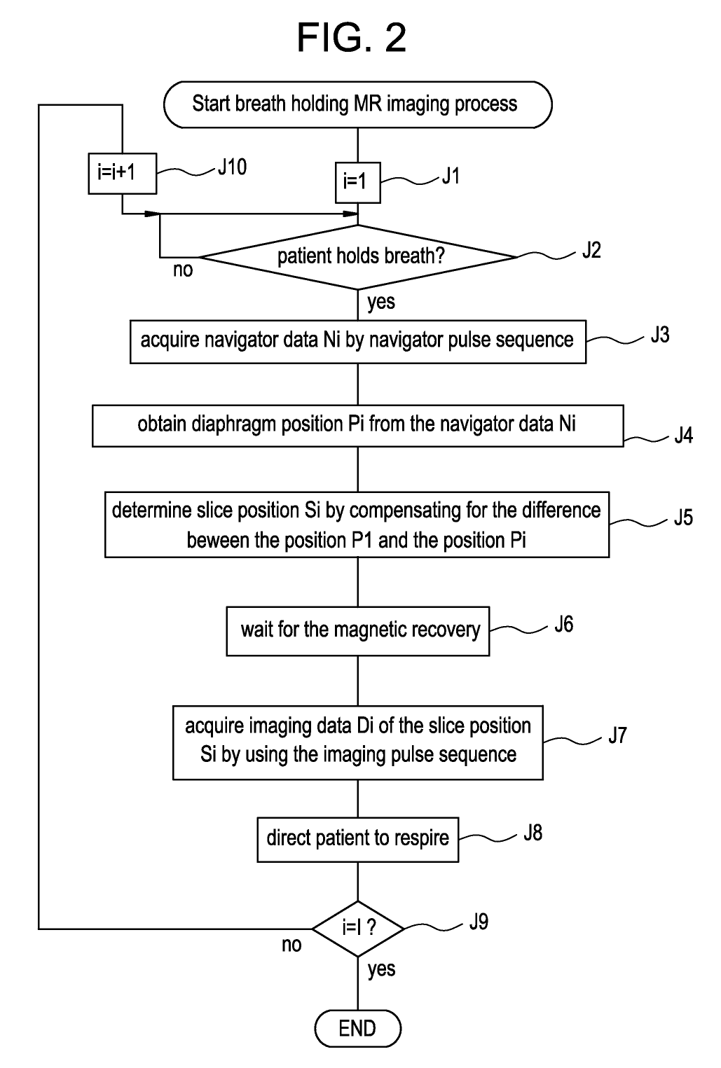

Now referring to FIG. 2, there is shown a flow diagram illustrating a breath holding MR imaging process in accordance with the first preferred embodiment of the present invention.

In step J1, the value in a repeat counter i is initialized to “1”.

In step J2, a patient is signaled to hold the breath, and the process proceeds to the next step J3 when the patient hold the breath.

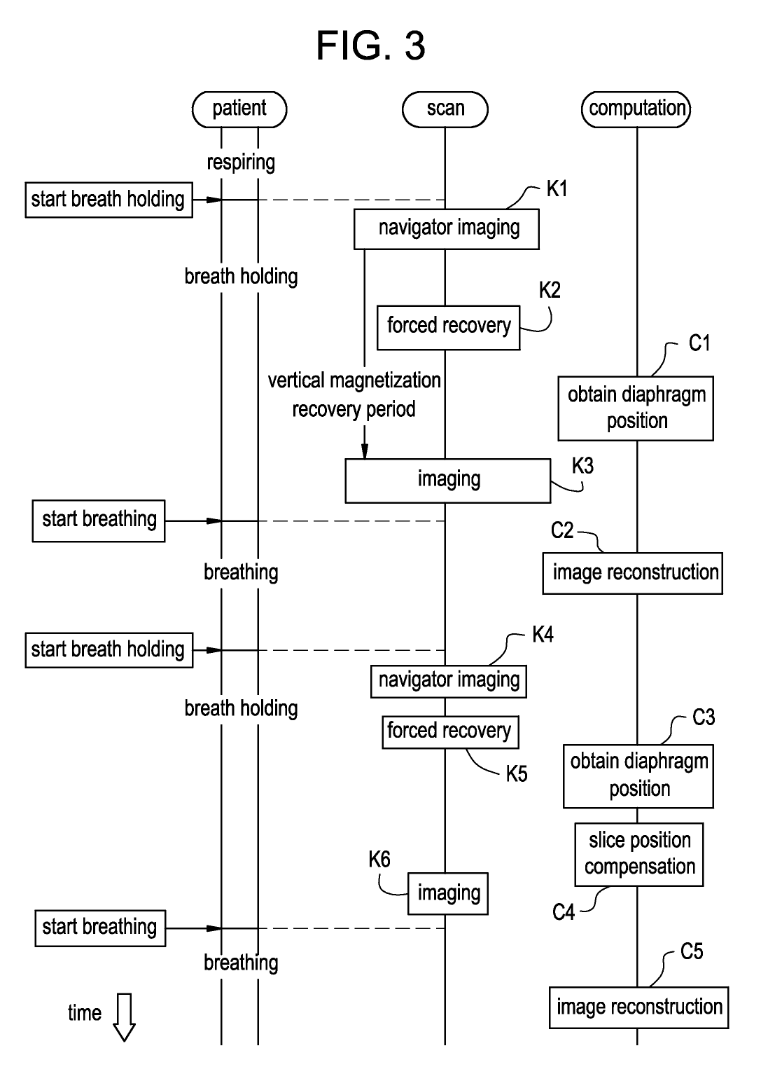

In step J3, a pulse sequence for the navigator is used to collect the data Ni for the navigator. The reference numeral K1 shown in FIG. 3 corresponds to step J3 for the first run (i=1), and K4 corresponds to step J3 for the second run (i=2).

The purpose of collecting navigator data Ni is to detect the diaphragm position Pi. In this description, for the sake of simplification, the imaging area is assumed to be in a shape of line extending along with the body axis. The navigator pulse sequence may use therefore a well known pulse sequence that can excite the imaging area in the shape of line. As the amount of displacement in the direction of body axis is approximately 2 cm, the length of the imaging area may be more than 2 cm in the direction of body axis.

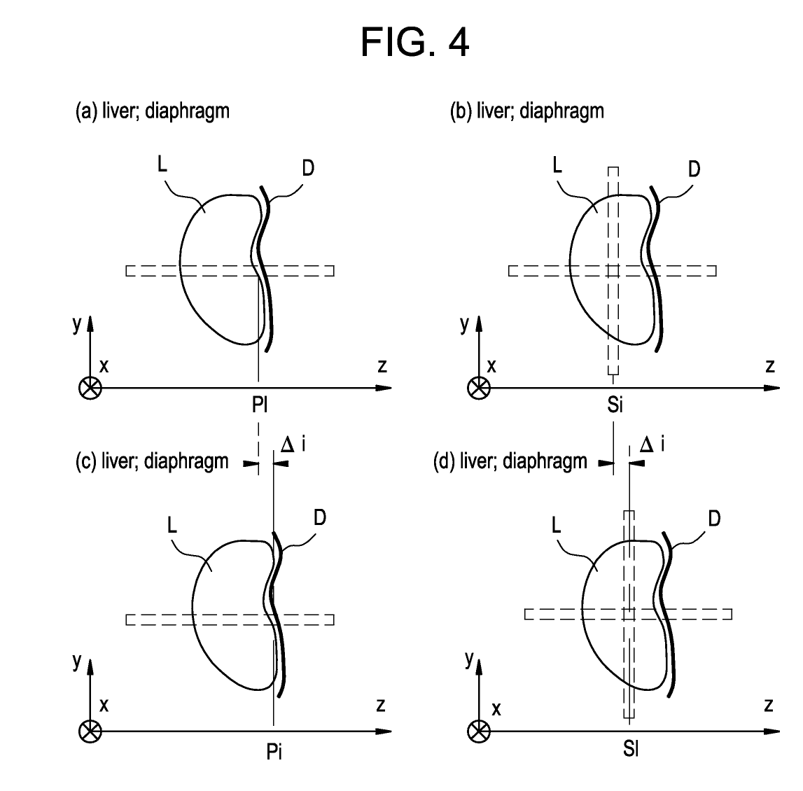

In step J4, the diaphragm position Pi is obtained from the navigator data Ni. The reference numeral C1 shown in FIG. 3 corresponds to step J4 in the first run (i=1), and the reference numeral C3 corresponds to step J4 of the second run (i=2). The reference numeral P1 shown in FIG. 4(a) indicates the position P1 of diaphragm D at the time of first run (i=1), and the reference numeral P2 shown in FIG. 4(c) indicates the position Pi of diaphragm D at the time of a run after the second (i>2).

In step J5, the difference A i between the position P1 of diaphragm D at the first run (i=1) and the position Pi of diaphragm D at the ith run (i≧1) is determined, the predefined slice position is compensated for by the difference Δ i to set the slice position S1 for the ith run. At the first run (i=1), the difference Δ1=0, so that the predefined slice position will become the slice position S1 for the first run. When the difference Δi≠0 at the time of a run after the second (i>2), the slice position will be compensated for. The reference numeral S1 shown in FIG. 4(b) indicates the slice position S1 at the first run (i=1), and the reference numeral Si shown in FIG. 4(d) indicates the slice position Si at the time of a run after the second (i>2).

In step J6, the process waits for the recovery of magnetization excited by the navigator pulse sequence to proceed to step J7. This waiting period is the vertical magnetization recovery period shown in FIG. 3. The vertical magnetization recovery period may be longer than one second, for example it can be two seconds. However, the vertical magnetization recovery period can be shortened or omitted if the residual lateral magnetized component is forcibly recovered to vertical magnetization by making use of one of well known fast recovery methods for example by applying fast recovery pulses such as K2 and K5 shown in FIG. 3. The vertical magnetization recovery time may also be shortened or omitted if the image capturing for the actual imaging uses the reverse centric view ordering, which suppress the banding artifacts as will be described below.

In step J7, imaging pulse sequence is used to collect the imaging data Di at the slice position Si. The reference numeral K3 shown in FIG. 3 corresponds to step J7 at the time of first run (i=1), and the reference numeral K6 corresponds to step J7 at the time of second run (i=2).

In step J8, the patient is directed to breathe, so as for the patient to respire.

In step J9, if the value of the counter i reaches to the planned number of repetition I, then the scan process will be terminated, otherwise if not then the process proceeds to step J10.

In step J10, the value of the counter i is incremented by ‘1’ and then the process proceeds back to step J2.

While the patient respires, images may be reconstructed as C2 and C5 shown in FIG. 3.

In accordance with the MRI apparatus 100 of the first preferred embodiment, the slice position can follow the positional displacement of the organ due to the timing difference of breath holding among runs. This may achieve following effects:

(1) In the image capturing for the actual imaging J7, when an abdominal contrast MR angiography is conducted to try to obtain a time intensity curve, a correct time intensity curve can be obtained because the imaging position of every run is the same with respect to the organ. Also when trying to obtain a subtraction image, a correct subtraction image can be obtained.

(2) In the image capturing for the actual imaging J7, when a multi-phase imaging of the liver L as shown in FIG. 4 (i.e., arterial phase, portal vein phase, and equilibrium phase), the comparison between phases can be correctly performed because the slice position Si of every run is the same with respect to the liver L.

(3) When the sensitivity compensation imaging using the body coil 101(0) in a first run of image capturing step for the actual image J7, and the image capturing for the actual imaging using the surface coils 101(1) . . . 101(T) in a second run of the image capturing step J7 for the actual imaging, the sensitivity compensation can be correctly performed because the slice position at the time of sensitivity compensation imaging and the slice position at the time of image capturing for the actual imaging are the same. This is especially effective for the imaging methods such as PURE and CLEAR.

(4) When an image is captured for the reference image in a first run of image capturing step for the actual imaging J7 and an image is captured for the parallel imaging in a second run and later of the image capturing step for the actual imaging J7, the parallel imaging can be correctly performed because the slice position for the reference image and the slice position for the parallel imaging are the same. This is especially effective for the imaging methods such as ASSET and SENSE.

(5) When images are captured for the image fusion while changing the imaging condition in the image capturing step J7 for the actual imaging, the slice position for every run is always the same so that the misregistration can be eliminated.

(6) When images are captured for the diffusion imaging with NEX increased in the image capturing step J7 for the actual imaging, the distortion in the diffusion image can be avoided.

### Second Embodiment

The present invention can be equally applied to any tomographic imaging apparatuses other than MRI apparatus.

0] Many widely different embodiments of the invention may be configured without departing from the spirit and the scope of the present invention. It should be understood that the present invention is not limited to the specific embodiments described in the specification, except as defined in the appended claims.

1. A breath holding MR imaging method, comprising:
an image capturing step for navigator for imaging an MR image having the imaging area including the diaphragm while holding breath;
a diaphragm position obtaining step for obtaining the position of diaphragm by analyzing said navigator image;
an image capturing step for the actual imaging for imaging an MR image of a desired slice while holding breath; and
a breath releasing interval step for releasing respiration,
wherein the steps are iteratively repeated two or more times in this order, and
wherein the slice position of second session or later is made to be a position which is set such that the slice position of first run is compensated for by the difference between the diaphragm position of the first run and the diaphragm position of the second run or later.

2. A breath holding MR imaging method according to claim 1, wherein the imaging area of the image capturing step for the navigator is in the form of a line extending along with the body axis.

3. A breath holding MR imaging method according to claim 1, wherein prior to said image capturing step for the actual imaging, a recovery step is provided for waiting for one second or more for the residual lateral magnetized component to be recovered to vertical magnetization.

4. A breath holding MR imaging method according to claim 1, wherein prior to said image capturing step for the actual imaging, a fast recovery step is provided for forcibly recovering the residual lateral magnetized component to vertical magnetization.

5. A breath holding MR imaging method according to claim 1, wherein said image capturing step for the navigator and said image capturing step for the actual imaging use the reverse centric view ordering.

6. A breath holding MR imaging method according to claim 1, wherein a contrast MR angiography of abdomen imaging is conducted in said image capturing step for the actual imaging.

7. A breath holding MR imaging method according to claim 1, wherein a multi-phase imaging of liver is conducted in said image capturing step for the actual imaging.

8. A breath holding MR imaging method according to claim 1, wherein a sensitivity compensation imaging is conducted using a body coil in a first run of said image capturing step for the actual imaging, and image capturing for the actual image is conducted in second run or later of said image capturing step for the actual imaging.

9. A breath holding MR imaging method according to claim 1, wherein an image is captured for a reference image in a first run of said image capturing step for the actual imaging, and an image is captured for the parallel imaging in a second or later run of said image capturing step for the actual imaging.

10. A breath holding MR imaging method according to claim 1, wherein an image capturing for an image fusion by varying the imaging condition for each time is conducted in said image capturing step for the actual imaging.

11. A breath holding MR imaging method according to claim 1, wherein an image capturing for a dispersion image is conducted in said image capturing step for the actual image.

12. An MRI apparatus, comprising:
an image capturing device for a navigator for capturing MR images having an image area including a diaphragm while holding the breath;
a diaphragm position obtaining device for obtaining the diaphragm position by analyzing said navigator image;
an image capturing device for the actual imaging for capturing MR images of a desired slice while holding the breath;
a controller device for repetitively operating said device for two or more times with a breathing interval therebetween; and
a compensator device for making the position, which is set such that the slice position of a first run is compensated for by the difference between the diaphragm position of the first run and the diaphragm position of the second run or later, to be the slice position of a second run or later.

13. An MRI apparatus according to claim 12, wherein the imaging area of said image capturing step for the navigator is in the form of a line extending along with the body axis.

14. An MRI apparatus according to claim 12, wherein a recovery time for waiting for one second or more for the residual lateral magnetized component to be recovered to vertical magnetization prior to operating said image capturing device for the actual imaging.

15. An MRI apparatus according to claim 12, wherein a fast recovery device for forcibly recovering the residual lateral magnetized component to vertical magnetization is operated prior to operating said image capturing device for the actual imaging.

16. An MRI apparatus according to claim 12, wherein said image capturing step for the navigator and said image capturing step for the actual imaging use the reverse centric view ordering.

17. An MRI apparatus, using the MRI apparatus according to claim 12, for conducting a contrast MR angiography of abdomen imaging.

18. A breath holding MR imaging method, using the MRI apparatus according to claim 12, for conducting the multi-phase image capturing of the liver.

19. A breath holding MR imaging method, using the MRI apparatus according to claim 12, for conducting a sensitivity compensation imaging using a body coil in a first run of said image capturing step for the actual imaging, and conducting image capturing for the actual image in second run or later of said image capturing step for the actual imaging.

20. A tomographic imaging apparatus, comprising:
an imaging device for a navigator which captures a tomographic image having an imaging area including the diaphragm in the direction of body axis while holding the breath;
a diaphragm position obtaining device for obtaining the diaphragm position by analyzing the navigator image;
an imaging device for the actual imaging for capturing a tomographic image of a desired slice while holding the breath;
a controller device for repetitively operating said device for twice or more with a breathing interval therebetween; and
a compensator device for making the position, which is set such that the slice position of a first run is compensated for by the difference between the diaphragm position of the first run and the diaphragm position of the second run or later, to be the slice position of a second run or later.
